(19) United States
(12) Patent Application Publication
(10) Pub. No.: US 2020/0121827 A1
(43) Pub. Date: Apr. 23, 2020

(54) BONE REGENERATION MATERIAL

(71) Applicant: Wishbone, Flemalle (BE)

(72) Inventors: Emilie DORY, Andenne (BE); Eric ROMPEN, Liège (BE); France LAMBERT, Trooz (BE); Geoffrey LECLOUX, Chaudfontaine (BE)

(73) Assignee: Wishbone, Flemalle (BE)

(21) Appl. No.: 16/477,904

(22) PCT Filed: Jan. 15, 2018
(86) PCT No.: PCT/EP2018/050877

§ 371 Date: Jul. 15, 2019

Related U.S. Application Data
(63) Continuation-in-part of application No. PCT/EP2017/050779, filed on Jan. 16, 2017.

(30) Foreign Application Priority Data
Jan. 16, 2017 (BE) BE2017/5025

(51) Int. Cl. A61L 27/36; A61L 27/12; A61L 27/56

(52) U.S. Cl. CPC A61L 27/365; A61L 27/56; A61L 27/3608; A61L 27/12

(57) ABSTRACT

The present invention relates to a bone regeneration material comprising: a solid first phase of hydroxyapatite of natural origin which is macroporous, having pores of diameters greater than or equal to 50 pm, preferably pores of diameters between 50 and 100 pm, and a solid synthetic second phase of calcium phosphate intended to enrich said first phase, said second phase having a Ca/P molar ratio of between 0.2 and 2, preferably of between 0.3 and 1.9, preferentially of between 0.5 and 1.65, said bone regeneration material having a defined weight ratio of said solid first phase of hydroxyapatite of natural origin to said solid synthetic second phase of calcium phosphate of between 99/1 and 1/99.

The present invention relates to a bone regeneration material comprising a first solid phase of hydroxyapatite of natural origin which is macroporous having pores of diameters greater than or equal to 50 μm, preferably pores of diameters of between 50 and 100 μm.

Such a bone regeneration material is known from document WO2015/049336 and is used as part of treating bone damage in different fields such as corrective or cosmetic surgery.

Hydroxyapatite is a mineral species from the phosphate family, of formula Cas(PO₄)₃(OH). More specifically, hydroxyapatite belongs to the apatite crystallographic family which are isomorphic compounds having one same hexagonal structure. This compound is widely used as a biomaterial for many years in different medical specialities, as hydroxyapatites are the most frequent crystalline calcium phosphates, and the first mineral constituents of bones, tooth enamel and dentine. Moreover, hydroxyapatites (and in particular, hydroxyapatites of natural origin) have good biocompatibility and specific adsorption properties of cells or proteins.

It is thus recognised that hydroxyapatite of natural origin has osteoconductive properties as well as a crystalline structure and a morphology identical to those of a natural bone material in humans, hydroxyapatite consequently perfectly suiting as part of a bone reconstructive or corrective surgery. In this sense, numerous materials comprising hydroxyapatite are currently used as dental implants to stimulate the bone reconstruction on bone sites having defects or damage. In particular, using a natural hydroxyapatite coming from bone samples cleaned of the organic substances thereof (proteins, prions, peptides and lipids) makes it possible to obtain a matrix, particularly specific to bone regeneration compared with the artificial analogue thereof. It is preferable that the bone regeneration material is purified beforehand of any trace of organic substances such that the implant is correctly accepted/integrated by the organism, is biocompatible and can interact by osteoconduction with the biological medium wherein it is placed.

As examples, hydroxyapatites can be used as substitution materials to replace or regenerate diseased or damaged tissues. They are also frequently used as a coating on titanium protheses to facilitate the osteointegration or also can prevent wear due to micromovements between the implant and the bone. Hydroxyapatites can also be used as cement in order to replace autogenic bone grafts. Furthermore, a growing number of hydroxyapatite applications as a medication vector is developed, this because hydroxyapatite has a structure with interconnected micropores.

As mentioned above, hydroxyapatite has a chemical composition very close to that of the mineral phase of the bone and the biological properties thereof as well as the biocompatibility thereof make it an excellent bone substitution product. It is specified that a bone colonisation depends on the porous characteristics of the bone regeneration material and the interconnection between the macropores thereof (number and dimension). These interconnections constitute types of “tunnels” which make it possible for the passage of cells and blood circulation between the pores thus encouraging bone formation.

Hydroxyapatite, through the high biocompatibility level thereof and the slow resorption thereof in the organism, can be administered in different forms and in numerous tissues for various applications. In particular, hydroxyapatite-based malleable cements are distinguished which are prepared then administered on the zone to be treated where they harden. These cements are used as a bone substitute due to the good osteointegration thereof and the bioresorbability thereof making it possible to give way to a neoformed bone over time.

There are also solutions and hydroxyapatite-based gels which can contain a polymer such as carboxymethylcellulose. Thus, bioactive ceramic-polymer composite implants are spoken of, these products can be used in different fields such as to fill periodontal deficiencies in dental surgery or carry out fillings in orthopaedic surgery.

Certain hydroxyapatite-based products are also presented in the form of a block, powder or granules used in orthopaedics, fillings or as a complement to dental implants.

Among hydroxyapatite-based products, numerous are those which are not ready for use and which require a preparation beforehand. It is the case, in particular, for hydroxyapatite-based cements which require a prior mixing of a powder and a solution before deposition on or in a zone to be treated where an in situ hardening is thus carried out. Other hydroxyapatite-based products also require to be shaped before implantation on or in the zone to be treated.

Unfortunately, even if such a bone regeneration material comprising a solid phase of hydroxyapatite of natural origin which is macroporous has properties specific to an endo-osseous implantation and makes it possible for a correct bone regeneration thanks to the biocompatibility thereof and to the osteoconductive capacities thereof, it is observed, in practice, that this type of bone regeneration material is not always optimal and that the timeframe for the bone regeneration process is always relatively long (1 mm/month). Moreover, the bone regeneration quantity is frequently insufficient, which leads to the formation of partial fibroses rather than a total ossification.

With the aim of accelerating the bone regeneration process, document US2002/0114755 discloses a material comprising hydroxyapatite and 50 to 90% by mass of tricalcium phosphate, the material according to this prior document being obtained by converting hard algal tissues in an aqueous phosphate solution with the addition of Mg⁺⁺ ions at an increased temperature.

The bone regeneration material according to this prior document, i.e. which comprises hydroxyapatite and 50 to 90% by mass of tricalcium phosphate of natural origin, has an interconnected porous structure, an improved and controllable resorption in the organism leading to an accelerated bone regeneration.

Unfortunately, even if a bone regeneration material according to document US2002/0114755 makes it possible to accelerate the bone regeneration process by increasing the calcium and phosphorus contribution in the form of free ions in the direct medium of the implantation site, it is observed that this type of material is not optimal.

Indeed, the method for obtaining such a bone regeneration material consists of transforming hydroxyapatite into tricalcium phosphate. Therefore, this is the conversion of a first natural phase into a second natural phase, hydroxyapatite being replaced with tricalcium phosphate.

Thus, during the implantation of the bone regeneration material according to document US2002/0114755, tricalcium phosphate which is more soluble than hydroxyapatite will quickly be solubilised and release calcium and phosphorous ions in the medium of the implantation site. As mentioned above, the tricalcium phosphate of such a bone regeneration material represents 50 to 90% by mass and is obtained by the conversion of a portion of hydroxyapatite contained in the starting material, namely the hard algal tissue. Consequently, with a bone regeneration material according to this prior document, 50 to 90% of the mass (corresponding to tricalcium phosphate) will quickly be solubilised following the implantation and thus lead to a natural hydroxyapatite material having wide empty zones and therefore to a hydroxyapatite material which is certainly of natural origin but which no longer has optimal topographic characteristics (porosity, specific surface area, etc.) making it possible for a good bone regeneration.

A bone regeneration material according to document US2002/0114755 therefore does not make it possible to maintain, over the long term, the suitable microporous structure of natural hydroxyapatite once this implanted and does not therefore make it possible to ensure an optimal bone regeneration process on the implantation site in terms of proliferation and differentiation of bone cells as such a material, once implanted, no longer has a superficial topography, nor a suitable microporosity.

The invention aims to overcome the disadvantages of the state of the art by providing a bone regeneration material making it possible to minimise the bone regeneration time while stimulating the bone regeneration potential so as to obtain an optimal regenerated bone quantity.

The bone regeneration material according to the invention not only has chemical properties (optimised calcium and phosphorus salting-out in the form of free ions) which make it possible to minimise the bone regeneration time, but also has optimal physical properties (topography and porosity conserved) enabling a good bone colonisation so as to obtain an optimal regenerated bone quantity.

To resolve this problem, a bone regeneration material according to the invention is provided, comprising a first solid phase of hydroxyapatite of natural origin which is macroporous having pores of diameters greater than or equal to 50 μm, preferably pores of diameters of between 50 and 100 μm, said bone regeneration material, the bone regeneration material according to the invention being characterised in that it further comprises a second solid synthetic phase of calcium phosphate intended to enrich said first phase, said second phase having a Ca/P molar ratio of between 0.2 and 2, preferably of between 0.3 and 1.8, preferably between 0.5 and 1.65, said bone regeneration material having a defined weight ratio between said first solid phase of hydroxyapatite of natural origin and said second solid synthetic phase of calcium phosphate of between 99/1 and 1/99.

Advantageously, according to the invention, the defined weight ratio between said first solid phase of hydroxyapatite of natural origin and said second solid synthetic phase of calcium phosphate is 95/5, 90/10, 85/15, 80/20, 75/25, 70/30, 65/35, 60/40, 55/45, 50/50, 45/55, 40/60, 35/65, 30/70, 25/75, 20/80, 15/85, 10/90 or 5/95.

In the scope of the present invention, it has been highlighted that such a bone regeneration material according to the invention having:

a first solid phase of hydroxyapatite of natural origin having pores of diameters greater than or equal to 50 μm, preferably pores of diameters of between 50 and 100 μm;

a second solid synthetic phase of calcium phosphate intended to enrich said first phase;

a Ca/P molar ratio for the second solid synthetic phase of calcium phosphate of between 0.2 and 2, preferably of between 0.3 and 1.8, preferably of between 0.5 and 1.65; and

a defined weight ratio between said first solid phase of hydroxyapatite of natural origin and said second synthetic solid phase of calcium phosphate of between 99/1 and 1/99, makes it possible to ensure a suitable salting-out of calcium (for example, in the form of extracellular free Ca²⁺ ions) and phosphorus (for example, in the form of extracellular free PO₄³⁻ ions) in the medium of the bone regeneration site such that these can play the role of promoters of the regrowth of surrounding biological tissues by significantly encouraging the proliferation and the differentiation of bone cells as well as mineralisation.

Furthermore, it has been observed, surprisingly, that the bone regeneration material according to the invention, i.e. of which the solid phase of hydroxyapatite is a natural phase of hydroxyapatite which is enriched by a second phase of calcium phosphate, has optimal topographic characteristics (porosity, specific surface area, etc.) making it possible to optimise the bone regeneration by enabling a good bone colonisation, and this even after the implantation of the bone regeneration material.

A good bone colonisation depends on the porous characteristics of the bone generation material and the interconnection between the macropores thereof (number and dimensions), these interconnections constituting types of “tunnels”, which make it possible for the passage of cells and blood circulation between the pores encouraging bone formation. The conservation of the topographic characteristics of natural hydroxyapatite is therefore paramount to obtain a suitable bone regeneration, which is not the case with a regeneration material according to document US2002/0114755 where a portion (50 to 90% by mass) of the natural phase of hydroxyapatite is converted into a natural phase of tricalcium phosphate.

A bone regeneration material according to the invention therefore has both optimal chemical and physical properties (optimised calcium and phosphorus salting-out in the form of free ions/topography, porosity) which ensure a bone regeneration and a formation of the bone optimised in a significantly reduced time with respect to a bone regeneration material such as that of documents WO2015/049336 and US2002/0114755.

In particular, in the scope of the present invention, it has been highlighted that, when the defined weight ratio between said first solid phase of hydroxyapatite of natural origin and said second synthetic solid phase of calcium phosphate is of between 99/1 and 1/99, and is preferably 95/5, 90/10, 85/15, 80/20, 75/25, 70/30, 65/35, 60/40, 55/45, 50/50, 45/55, 40/60, 35/65, 30/70, 25/75, 20/80, 15/85, 10/90 or 5/95, the bone regeneration time is significantly reduced. This is due to the fact that with such a ratio, at least one of the calcium and/or phosphorus salting-outs in the form of free ions is such that calcium and/or phosphorus is thus located in the surrounding bone regeneration medium in a quantity making it possible to significantly increase the concentration here with respect to the concentration naturally encountered, while only hydroxyapatite tends to capture calcium and phosphorus in the form of free ions and to deplete the surrounding medium thereof from the bone regeneration site.

It must be noted, that two-phase bone regeneration materials are described in the state of the art, in particular materials consisting of a synthetic hydroxyapatite and synthetic tricalcium calcium phosphate association: these are therefore 100% synthetic materials which, as for a regeneration material such as indicated at the start, impose a relatively long bone regeneration duration. Moreover, such totally synthetic two-phase bone regeneration materials do not make it possible, on the contrary, for a material according to the invention, to ensure an optimal bone regeneration on the implantation site in terms of proliferation and differentiation of bone cells as they do not have a suitable superficial topography nor a suitable microporosity.

Preferably, according to the invention, said second synthetic solid phase of calcium phosphate has a Ks solubility product greater than that of said first phase of hydroxyapatite of natural origin. This is particularly advantageous, since calcium and phosphorus must be contributed in the form of free ions in the direct medium of the implantation site while conserving the structural properties (pore size, specific surface area, etc.) and composition of the solid phase of hydroxyapatite of natural origin. Since the solubility of the second phase is greater, it is mainly this phase in particular which will be at the origin of the calcium and phosphorus release in the form of free ions (for example, Ca²⁺ and PO₄³⁻) and not hydroxyapatite of natural origin, which is clearly less dissolved and which, on the contrary, tends to fix these ions at the start of the implantation medium.

Advantageously, according to the invention, said first solid phase of hydroxyapatite of natural origin has a specific surface area greater than 4m²/g.

Preferably, according to the invention, said second synthetic solid phase of calcium phosphate is selected from the group constituted of monocalcium calcium phosphate (MCP), dicalcium calcium phosphate (DCP), octacalcium phosphate (OCP), calcium deficient apatite (CDA), amorphous calcium phosphate (ACP), tricalcium calcium phosphate (TCP), tetracalcium calcium phosphate (TTCP), and the mixtures thereof.

Advantageously, according to the invention, said first phase of hydroxyapatite of natural origin is hydroxyapatite obtained from a bone material of natural origin, in particular from a bone material of animal origin. For example, hydroxyapatite of natural origin can be hydroxyapatite obtained from a bone material coming from a mammal (bovine, horse, pig or sheep), coral or dry.

Preferably, according to the invention, said hydroxyapatite of natural origin which is macroporous of said first phase is a hydroxyapatite of natural origin which is macroporous at least partially sintered. The controlled sintering (heating of a powder without leading it to melt) advantageously makes it possible to obtain a hydroxyapatite of natural origin having increased mechanical resistance properties while conserving a suitable microporosity and a suitable surface topography, which makes it possible to facilitate the implantation and to improve the long-term stability after implantation.

Advantageously, the bone regeneration material according to the invention, further comprises at least one therapeutic agent selected in the group constituted of antibiotics, antivirals, anti-inflammatories, hormones such as steroids, growth factors such as BMPs (Bone Morphogenetic Proteins), anti-rejection agents, stem cells, and the mixtures thereof.

Preferably, the bone regeneration material according to the invention is intended to be used as an implant or prothesis for a bone formation, a bone regeneration or for a bone correction in a mammal, preferably in a human.

Other embodiments of a bone regeneration material according to the invention are indicated in the appended claims.

The inventions also aims for a medical device containing a bone regeneration material according to the invention.

Other embodiments of a medical device according to the invention are indicated in the appended claims.

The invention also aims for a bone regeneration composition comprising a bone regeneration material according to the invention.

Other embodiments of a composition according to the invention are indicated in the appended claims.

The present invention is also based on a method for enriching a bone regeneration material according to the invention, said method comprising:

a step of supplying a bone regeneration material, and

a step of enriching said bone regeneration material, in calcium and in phosphorus,

said method being characterised in that said calcium and phosphorus enriching step is carried out by at least one first and at least one second separate soaking succeeding one another in any order, said at least one first soaking taking place in a first solution comprising calcium and said at least one second soaking taking place in a second solution comprising phosphorus.

Preferably, according to the method according to the invention, said first soaking takes place in a first solution of Ca(NO₃)₂.4H₂O, of CaCl₂.2H₂O, of Ca(OH)₂, CaSO₄.2H₂O, or of CaCO₃.

Advantageously, according to the method according to the invention, said second soaking takes place in a second solution of Na₃PO₄, of Na₂HPO₄, of NaH₂PO₄.H₂O, of K₃PO₄ of K₂HPO₄, of KH₂PO₄, of K₂HPO₄, of (NH₄)₃PO₄, of (NH₄)₂HPO₄ or of NH₄H₂PO₄.

Other embodiments of a method according to the invention are indicated in the appended claims.

Other characteristics, details and advantages of the invention will emerge from the description given below, in a non-limiting manner and by making reference to the appended figures.

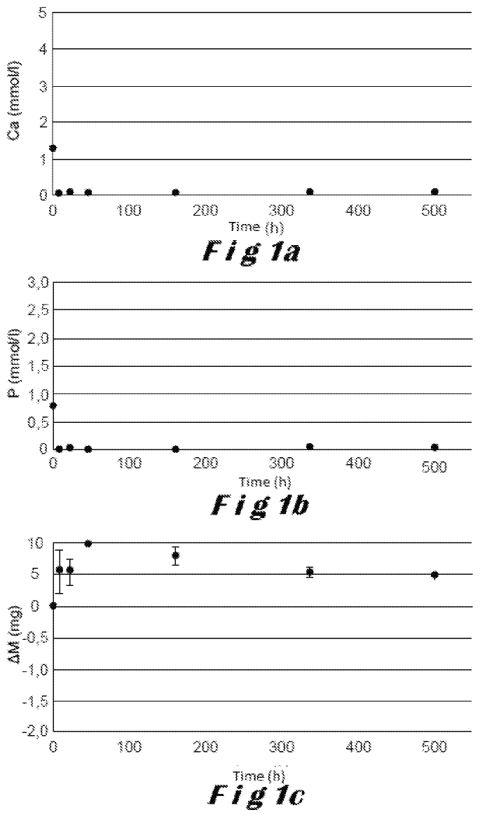

FIGS. 1a, 1b and 1c illustrate respectively the development of the calcium (Ca) concentration over time for a solid phase of hydroxyapatite of natural bovine origin, non-enriched, immersed in an HBSS medium, the development of the phosphorus (P) concentration over time for a solid phase of hydroxyapatite of natural bovine origin, non-enriched, immersed in an HBSS medium, and the weight increase over time for a solid phase of hydroxyapatite of natural bovine origin, non-enriched, immersed in an HBSS medium.

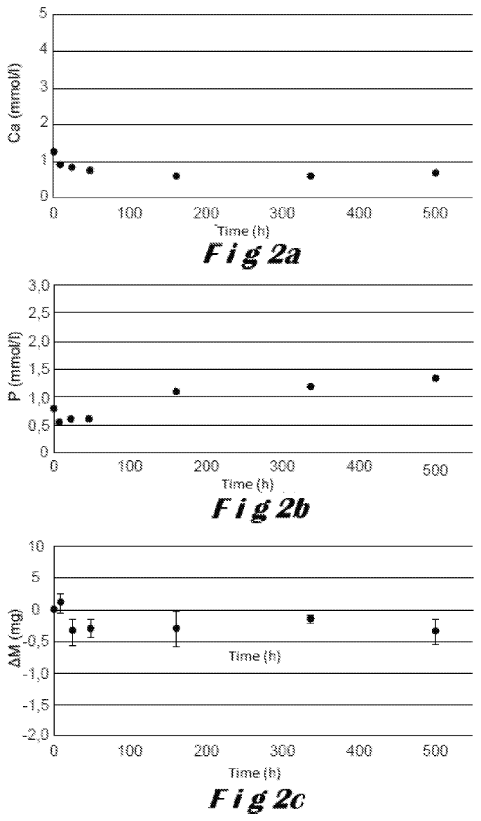

FIGS. 2a, 2b and 2c illustrate respectively the development of the calcium (Ca) concentration over time for a material immersed in an HBSS medium and comprising a solid phase of hydroxyapatite of natural bovine origin enriched by a synthetic solid phase of calcium phosphate (ratio 90/10), the development of the phosphorus (P) concentration over time for a material immersed in an HBSS medium and comprising a solid phase of hydroxyapatite of natural bovine origin enriched by a synthetic solid phase of calcium phosphate (ratio 90/10) and the weight increase for a material immersed in an HBSS medium and comprising a solid phase of hydroxyapatite of natural bovine origin enriched by a synthetic solid phase of calcium phosphate (ratio 90/10).

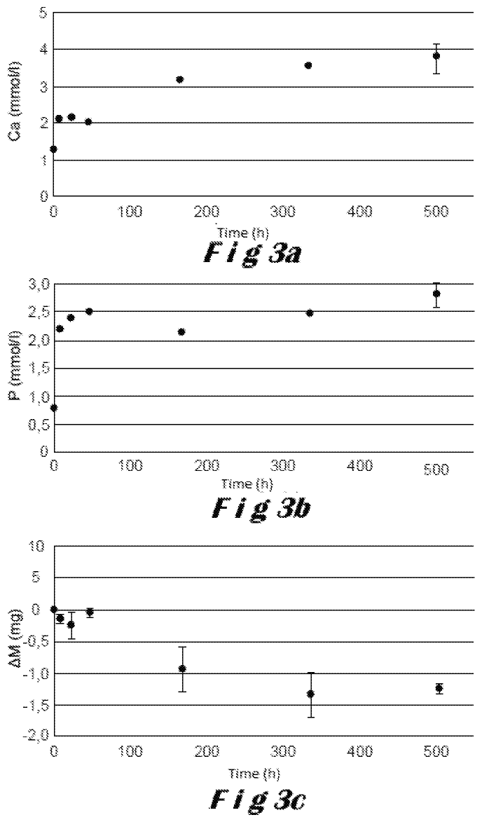

FIGS. 3a, 3b and 3c illustrate respectively the development of the calcium (Ca) concentration over time for a material immersed in an HBSS medium and comprising a solid phase of hydroxyapatite of natural bovine origin enriched by a synthetic solid phase of calcium phosphate (ratio 80/20), the development of the phosphorus (P) concentration over time for a material immersed in an HBSS medium and comprising a solid phase of hydroxyapatite of natural bovine origin enriched by a synthetic solid phase of calcium phosphate (ratio 80/20) and the weight increase for a material immersed in an HBSS medium and comprising a solid phase of hydroxyapatite of natural bovine origin enriched by a synthetic solid phase of calcium phosphate (ratio 80/20).

### EXAMPLES

Comparison of Calcium and Phosphorus Salting-Outs in the Form of Free Ions Over Time for Different Bone Regeneration Materials—Solubility Tests

Comparative tests have been carried out in order to determine the calcium and phosphorus quantities (concentrations) salted-out over time (solubility tests) at the start of different bone regeneration materials, this in an medium reproducing the in vivo implantation conditions.

### 1. Methodology

To do this, the calcium and phosphorus ratios and quantities (concentrations) salted-out have been measured in a medium simulating the in vivo implantation conditions (human blood plasma), i.e. in an HBSS medium (Hank's balanced salt medium) having a pH of 7 and of which the composition is outlined in table 1 below:

TABLE 1

| | Concentrations (mM) |
|---|---|
| Na⁺ | 142.8 |
| K⁺ | 5.8 |
| Mg²⁺ | 0.9 |
| Ca²⁺ | 1.3 |
| Cl⁻ | 146.8 |
| HCO³⁻ | 4.2 |
| HPO₄²⁻ | 0.3 |
| H₂PO⁴⁻ | 0.4 |
| SO₄²⁻ | 0.4 |
| Glucose | 5.6 |

The following bone regeneration materials have been tested:

Material 1: 0.5 g of solid phase of hydroxyapatite of natural bovine origin, non-enriched, having pores of a size of between 50 and 100 μm;

Material 2: 0.5 g of solid phase of hydroxyapatite of natural bovine origin having pores of a size of between 50 and 100 μm and enriched by a synthetic solid phase of calcium phosphate according to a ratio by weight of solid phase of hydroxyapatite/synthetic solid phase of calcium phosphate of 90/10, the phase of calcium phosphate having a Ca/P molar ratio of less than 1.67; and

Material 3: 0.5 g of solid phase of hydroxyapatite of natural bovine origin having pores of a size of between 50 and 100 μm and enriched by a synthetic solid phase of calcium phosphate according to a ratio by weight of solid phase of hydroxyapatite/synthetic solid phase of calcium phosphate of 80/20, the phase of calcium phosphate having a Ca/P molar ratio of less than 1.67.

The solid phase of hydroxyapatite of natural bovine origin is obtained according to the method described in document WO2015/049336 and is more specifically composed of hydroxyapatite particles having a size of between 0.25 mm and 1 mm.

The materials comprising a first solid phase of hydroxyapatite of natural origin and a second synthetic solid phase of calcium phosphate have been obtained by successive and alternate soakings for a duration of 5 minutes of the first solid phase of hydroxyapatite of natural origin (hydroxyapatite particles) in separate baths of Ca(NO₃)₂.4H₂O (1M, pH=10) and of NaH₂PO₄.H₂O (0.5M, pH=10). The first bath makes it possible to enrich the hydroxyapatite calcium (Ca²⁺) particles of natural origin, the second phosphorus (PO₄³⁻) bath.

Following 4 successive soakings (2 soakings in each bath) to obtain a ratio by weight of solid phase of hydroxyapatite/synthetic solid phase of calcium phosphate of 90/10 or following 6 successive soakings (3 soakings in each bath) to obtain a ratio by weight of solid phase of hydroxyapatite/synthetic solid phase of calcium phosphate of 80/20, the hydroxyapatite particles of natural origin enriched in calcium and in phosphorus have been rinsed in order to remove the excess calcium and phosphorus then dried at a temperature of 100° C. for 6 hours.

A total of 36 samples (of 0.5 g) have been tested simultaneously by immersing in 10 ml of HBSS medium at a temperature of 37° C. Calcium and phosphorus salting-out measurements in the form of free ions have been taken after 8 hours, 24 hours, 48 hours, 1 week, 2 weeks and 3 weeks on two samples for each material and by duration, these samples being rinsed and dried at a temperature of 100° C. after each of these durations.

The calcium and phosphorus quantities (concentrations) salted-out at the start of each sample in the HBSS medium have been measured according to the ICP-AES (Inductively Coupled Plasma—Atomic Emission Spectroscopy) technique, this following a removal of possible materials suspended in the HBSS medium by centrifugation. The weight of the materials have been defined by weighing (gravimetry) before and after immersion in the HBSS medium, this for each of the durations mentioned above.

### 2. Results

The results obtained are illustrated in FIGS. 1 to 3. For each of the materials (samples), the starting calcium and phosphorus concentrations are respectively 1.26 mmol/l and 0.78 mmol/l, which corresponds to the concentrations of these ions in the initial HBSS medium.

These results show that, when the material 1—solid phase of hydroxyapatite of natural bovine origin, non-enriched—is immersed in the HBSS medium, the calcium and phosphorus concentrations in the form of free ions decrease before being stabilised (FIGS. 1a and 1b). At the same time, an increase by weight of the material 1 is observed (FIG. 1c). For example, after 3 weeks, the calcium concentration in the medium is 0.06 mmol/l while the phosphorus concentration in the medium is 0.03 mmol/l for an average weight increase per sample of 5 mg.

For the material 2—solid phase of hydroxyapatite of natural bovine origin enriched by a synthetic solid calcium phase (ratio 90/10)—the calcium and phosphorus concentrations start by decreasing with a quick stabilisation of the calcium concentration (0.6 mmol/l) while the phosphorus concentration progressively increases up to reaching a final value of 1.34 mmol/l after 3 weeks of immersion (FIGS. 2a and 2b), this concentration being greater than the initial phosphorus concentration in the HBSS medium. The weights of the samples of the material 2 have first barely increased before decreasing down to a loss by weight of 3.5 mg (FIG. 2c).

Concerning the material 3—solid phase of hydroxyapatite of natural bovine origin enriched by a synthetic solid phase of calcium phosphate (ratio 80/20)—the calcium and phosphorus concentrations increase over time while the weights of the samples decreases (FIGS. 3a to 3c). The final calcium and phosphorus concentrations, after 3 weeks of immersion in the HBSS medium, are respectively 3.69 mmol/l and 2.78 mmol/l for a loss by weight of 12.5 mg, these concentrations being greater than those initially measured in the HBSS medium.

### 3. Conclusion

The results obtained with the material 1—solid phase of hydroxyapatite of natural bovine origin, non-enriched—highlight the capacity of hydroxyapatite to fix the calcium and the phosphorus in the form of free ions present in the HBSS medium to form an apatite layer on the surface thereof. Already after 8 hours of immersion, most of the calcium and phosphorus free ions are absorbed on the surface of the samples. These results correspond to the gravimetric recordings which indicate that the samples are heavier after 3 weeks of immersion. These results therefore indicate that the material 1 (non-enriched hydroxyapatite) fixes the calcium and the phosphorus in the form of free ions, and that the dissolution of hydroxyapatite in the HBSS medium is marginal.

These results show, for the material 2—solid phase of hydroxyapatite of natural bovine origin enriched by a synthetic solid phase of calcium phosphate (ratio 90/10)—that two phenomena take place concurrently since the calcium and phosphorus concentrations first both decrease, which is again linked to the capacity that hydroxyapatite has to fix these ions. However, the phosphorus concentration in the medium then increases and even exceeds the concentration of this compound in the initial HBSS medium, while the calcium concentration remains less than that initially measured in the HBSS medium by however being greater than that measured when the material 1 is immersed in this same medium. At the same time, after 3 weeks, the gravimetric results show that the samples have lost weight. All of these results for the material 2 indicate that this material is dissolved in the HBSS medium, which can only be attributed to the enriching phase (synthetic solid phase of calcium phosphate), since the results obtained with the material 1 show that the dissolution of hydroxyapatite under the same conditions is totally marginal.

The results obtained with the material 3—solid phase of hydroxyapatite of natural bovine origin enriched by a synthetic solid phase of calcium phosphate (ratio 80/20)—demonstrate that no decrease in calcium and phosphorus concentrations takes place, that on the contrary, since the calcium and phosphorus concentrations increase over time at the same time with a loss of weight of the samples. From these observations, it can be concluded that these increases of calcium and phosphorus concentrations are due to a dissolution of the second phase of the material 3 in the HBSS medium since the results obtained with the material 1 show that the dissolution of hydroxyapatite under the same conditions is totally marginal. Moreover, it can be observed that the material 3 does not fix the calcium and the phosphorus in the form of free ions salted-out or only marginally, which ensures that the latter remain in solution.

All of these results also show that the enriching (synthetic solid phase of calcium phosphate) is more soluble than hydroxyapatite and that it is able to salt-out the calcium and the phosphorus in the form of free ions (Ca²⁺ and PO₄³⁻) in the surrounding medium (HBSS imitating blood plasma).

It is well understood that the present invention is, in no manner, limited to the embodiments described above, and that modifications can be contributed to it without moving away from the scope of the appended claims.

1. Bone regeneration material comprising a first solid phase of hydroxyapatite of natural origin which is macroporous having pores of diameters greater than or equal to 50 μm, preferably pores of diameters of between 50 and 100 μm, said bone regeneration material being characterised in that it further comprises a second synthetic solid phase of calcium phosphate intended to enrich said first phase, said second phase having a Ca/P molar ratio of between 0.2 and 2, preferably of between 0.3 and 1.8, preferably of between 0.5 and 1.65, said bone regeneration material having a defined weight ratio between said first solid phase of hydroxyapatite of natural origin and said second synthetic solid phase of calcium phosphate of between 99/1 and 1/99.

2. Bone regeneration material according to claim 1, characterised in that said second synthetic solid phase of calcium phosphate has a Ks solubility product greater than that of said first phase of hydroxyapatite of natural origin.

3. Bone regeneration material according to claim 1, characterised in that said first solid phase of hydroxyapatite of natural origin has a specific surface area of greater than 4m²/g.

4. Bone regeneration material according to claim 1, characterised in that said second synthetic solid phase of calcium phosphate is selected from the group constituted of monocalcium calcium phosphate (MCP), dicalcium calcium phosphate (DCP), octacalcium phosphate (OCP), calcium deficient apatite (CDA), amorphous calcium phosphate (ACP), tricalcium calcium phosphate (TCP), tetracalcium calcium phosphate (TTCP), and the mixtures thereof.

5. Bone regeneration material according to claim 1, wherein said first phase of hydroxyapatite of natural origin is hydroxyapatite obtained from a bone material of natural origin, in particular from a bone material of animal origin.

6. Bone regeneration material according to claim 1, wherein said hydroxyapatite of natural origin which is macroporous of said first phase is a hydroxyapatite of natural origin which is macroporous at least partially sintered.

7. Bone regeneration material according to claim 1, further comprising at least one therapeutic agent selected from the group constituted of antibiotics, antivirals, anti-inflammatories, hormones such as steroids, growth factors such as BMPs (Bone Morphogenetic Proteins), anti-rejection agents, stem cells, and the mixtures thereof.

8. Bone regeneration material according to claim 1, intended to be used as an implant or prothesis for a bone formation, a bone regeneration or for a bone correction in a mammal, preferably in a human.

9. Medical device containing a bone regeneration material according to claim 1.

10. Bone regeneration composition comprising a bone regeneration material according to claim 1.

11. Method for enriching a bone regeneration material according claim 1, said method comprising:
a step of supplying a bone regeneration material, and
a step of enriching said bone regeneration material, in calcium and in phosphorus,
said method being characterised in that said calcium and phosphorus enriching step is carried out by at least one first and at least one second separate soaking succeeding one another in any order, said at least one first soaking taking place in a first solution comprising calcium and said at least one second soaking taking place in a second solution comprising phosphorus.

12. Method for enriching a bone regeneration material according to claim 11, characterised in that said first soaking takes place in a first solution of Ca(NO₃)₂.4H₂O, of CaCl₂.2H₂O, of Ca(OH)₂, CaSO₄.2H₂O, or of CaCO₃.

13. Method for enriching a bone regeneration material according to claim 11, characterised in that said second soaking takes place in a second solution of Na₃PO₄, of Na₂HPO₄, of NaH₂PO₄.H₂O, of K₃PO₄ of K₂HPO₄, of KH₂PO₄, of K₂HPO₄, of (NH₄)₃PO₄, of (NH₄)₂HPO₄ or of NH₄H₂PO₄.
